(19) United States
(12) Patent Application Publication
(10) Pub. No.: US 2009/0193846 A1
(43) Pub. Date: Aug. 6, 2009

(54) Recovery of Carbon Monoxide and Hydrogen From Hydrocarbon Streams

(76) Inventors: Michael J. Foral, Aurora, IL (US); Rian Reyneke, Katy, TX (US); Wayne W.Y. Eng, League City, TX (US); Graeme Parker, Falkirk (GB)

Correspondence Address: NIXON & VANDERHYE, PC, 901 NORTH GLEBE ROAD, 11TH FLOOR, ARLINGTON, VA, 22203, US

(21) Appl. No.: 11/922,951

(22) PCT Filed: Jul. 28, 2005
(86) PCT No.: PCT/US2005/026985

§ 371 Date: Dec. 27, 2007

(51) Int. Cl. F25J 3/02; C07C 7/04

(52) U.S. Cl. 62/620

(57) ABSTRACT

A process is described for the recovery of CO and optionally hydrogen from a stream containing CO, H2, methane, and hydrocarbons heavier than methane. The process is characterized by a two-stage removal (3, 6) of C2+ hydrocarbons from the feed. In a first step the feed gas (1) is separated (3) into a first C2+ depleted stream (5) and a first C2+ enriched stream (4). The first C2-enriched stream (4) is rectified (6) to produce a second C2+ depleted stream. The first and second C2+ depleted streams (5, 9) are fed to a cryogenic system (10) fro recovery of CO (12) and optionally hydrogen (11).

This invention was made with government support under United States Department of Energy Cooperative Agreement No. DE-FC07-01ID-14090.

The present invention relates to a process for the recovery of carbon monoxide, and optionally hydrogen, from a process stream comprising carbon monoxide, hydrogen, methane, and hydrocarbons heavier than methane (i.e. having two or more carbon atoms).

Carbon monoxide and hydrogen may be formed in a number of processes, including the endothermic steam reforming of hydrocarbons, particularly methane or naphtha-range hydrocarbons. The hydrogen and carbon monoxide may be obtained as a gas mixture comprising the hydrogen, carbon monoxide and, typically, methane, which can then be treated to recover separate hydrogen-rich and carbon monoxide-rich products. In commercial-scale operations cryogenic distillation is most often used to recover and purify the hydrogen and carbon monoxide products, though pressure swing adsorption techniques can also be employed.

Many cryogenic systems for the recovery of carbon monoxide and hydrogen from mixed gases utilize a methane wash tower in which the carbon monoxide and hydrogen-containing gas mixture (process stream) is contacted in a trayed or packed tower with a liquid methane-rich stream. Much of the carbon monoxide and methane contained in the gas mixture is dissolved into the methane-rich adsorbent to produce a bottoms liquid containing primarily carbon monoxide, methane, and dissolved hydrogen, and an overhead product containing primarily hydrogen and residual amounts of carbon monoxide and methane. The bottoms liquid is typically stripped of hydrogen in one or more stripping columns and the resulting stripped liquid is separated in a cryogenic carbon monoxide purification column into a (typically) gaseous carbon monoxide overhead product and a liquid methane bottoms product. Such processes are described, for example, in U.S. Pat. No. 4,888,035, U.S. Pat. No. 5,133,793, U.S. Pat. No. 5,592,831, U.S. Pat. No. 6,082,134, U.S. Pat. No. 6,269,657 and US 2002/0134243.

Other cryogenic systems for the recovery of carbon monoxide and hydrogen, which do not utilize a methane wash column but rely on partial condensation and stripping steps, are described, for example, in U.S. Pat. No. 5,509,271 and U.S. Pat. No. 6,173,585.

None of the above documents address the issues of recovery of carbon monoxide and hydrogen from a gas mixture comprising hydrocarbons heavier than methane (i.e. having 2 or more carbon atoms). Process streams comprising hydrocarbons with 2 or more carbons atoms may be obtained, for example, where carbon monoxide and hydrogen-containing gas mixtures arise from other sources, such as refinery off-gases and cracked gases produced during the production of light olefins via oxidative dehydrogenation. It is advantageous to remove at least some of these hydrocarbons with 2 or more carbons atoms from the process stream before it is charged to the carbon monoxide and hydrogen recovery process. The removal of the hydrocarbons with 2 or more carbons atoms from the cryogenic recovery process improves the energy efficiency of the process and can also reduce the risk of foaming in the cryogenic distillation system. In addition, the hydrocarbons with 2 or more carbons atoms themselves can be more valuable as a purified product than they would be as fuel stream, for example, if they were mixed with methane and burned.

U.S. Pat. No. 6,578,377 teaches a process for recovering hydrogen and carbon monoxide from a gas mixture containing carbon monoxide, hydrogen, methane, and hydrocarbons heavier than methane (hydrocarbons with 2 or more carbons atoms). The process comprises a step for separating the gas mixture into a stream depleted in hydrocarbons with 2 or more carbons atoms and a reject stream enriched in hydrocarbons with 2 or more carbons atoms, sending the stream depleted in hydrocarbons with 2 or more carbons atoms into a methane wash tower, recovering a CO-enriched overhead product and a methane-enriched liquid bottoms product from a cryogenic distillation system, and using at least a portion of the methane-enriched liquid to provide the methane-rich reflux to the methane wash column.

The partial condensation method of U.S. Pat. No. 6,578,377 concentrates the hydrocarbons with 2 or more carbons atoms in the reject liquid phase so that the molar ratio of hydrocarbons with 2 or more carbons atoms to methane in the gas phase is less than 0.05, preferably less than 0.02. However, this process has the disadvantage that such a process is relatively inefficient in separation of the carbon monoxide and hydrocarbons with 2 or more carbons atoms. Thus, as the temperature of the partial condensation decreases, the amount of hydrocarbons with 2 or more carbons atoms in the uncondensed vapor decreases, but also the recovery of carbon monoxide to the uncondensed vapor decreases.

The present invention provides an improved (i.e. more efficient) process for separating carbon monoxide from hydrocarbons with 2 or more carbons atoms in a mixed gas feed which contains carbon monoxide, hydrogen, methane, and hydrocarbons with 2 or more carbons atoms, which process allows a larger fraction of the carbon monoxide to enter a CO recovery process while at the same time preventing hydrocarbons with 2 or more carbons atoms from entering the process.

In a first aspect, the present invention provides a process for the separation of carbon monoxide from a process stream comprising carbon monoxide, hydrogen, methane and hydrocarbons with 2 or more carbons atoms, said process comprising:
a) passing the process stream to a first separation step to separate the process stream into a first intermediate stream comprising hydrocarbons with 2 or more carbons atoms and a reduced quantity of carbon monoxide, hydrogen and methane, and a second intermediate stream comprising carbon monoxide, hydrogen and methane and a reduced quantity of hydrocarbons with 2 or more carbons atoms,
b) passing the first intermediate stream to a second separation step to separate the first intermediate stream into a product stream comprising hydrocarbons with 2 or more carbons atoms and a third intermediate stream comprising carbon monoxide, hydrogen and methane, and
c) treating both the second and third intermediate streams in a cryogenic separation system to separate the carbon monoxide, and optionally the hydrogen, therein.

Steps (a) and (b) of the process of the present invention provide a “two-stage” separation for the separation of hydrocarbons with 2 or more carbon atoms from the methane, carbon monoxide and hydrogen components of the mixed gas feed which allows a high degree of separation between the hydrocarbons with 2 or more carbons atoms and the remaining carbon monoxide, hydrogen and methane mixture to be achieved. Thus, the present invention provides a higher recovery of carbon monoxide and higher recovery of the hydrocarbons with 2 or more carbons atoms as a product stream comprising hydrocarbons with 2 or more carbons atoms (“heavy” hydrocarbon stream). In a preferred embodiment of the present invention hydrogen and methane containing streams may be produced, giving four distinct products that may be produced: a purified carbon monoxide stream, a purified hydrogen stream, a purified methane stream, and a product stream comprising hydrocarbons with 2 or more carbons atoms. For the purposes of this invention, a purified carbon monoxide stream is considered to be one in which the concentration of carbon monoxide is at least 90 mol %, the concentration of hydrogen is less than 2 mol %, and the concentration of methane is less than 10 mol %. A purified methane stream is considered to be one in which the concentration of methane is at least 85 mol % and the concentration of components heavier than methane is less than 5 mol %. A purified hydrogen stream is considered to be one in which the concentration of hydrogen is at least 80 mol %.

Step (a) of the process of the present invention comprises passing the process stream comprising carbon monoxide, hydrogen, methane and hydrocarbons with 2 or more carbon atoms to a first separation step to separate the process stream into a first intermediate stream comprising hydrocarbons with 2 or more carbons atoms and a reduced quantity of carbon monoxide, hydrogen and methane, and a second intermediate stream comprising carbon monoxide, hydrogen and methane and a reduced quantity of hydrocarbons with 2 or more carbons atoms.

The first separation step preferably comprises partial condensation of the process stream, for example by cooling in a heat exchanger, followed by vapour/liquid separation in a flash drum or in a rectification column (in which the vapour is purified or enriched in carbon monoxide, hydrogen and methane by contact with a counterflowing stream of liquid condensed from the vapour).

Alternatively, the first separation step may comprise a combined rectification and chilling process, such as a dephlegmator, a distillation column with side condensers or an advanced heat integrated rectifier system, such as described in U.S. Pat. No. 6,343,487.

Step (a) of the process of the present invention produces a first intermediate stream comprising hydrocarbons with 2 or more carbons atoms and a reduced quantity of carbon monoxide. The amount of separation required in step (a) will depend on the actual amount of hydrocarbons with 2 or more carbons atoms in the process stream to step (a), but, in general, the first intermediate stream typically comprises at least 75% by mole of the hydrocarbons with two or more carbon atoms that were present in the initial process stream, such as at least 90% by mole of the hydrocarbons with 2 or more carbons atoms. Higher percentages of the hydrocarbons with 2 or more carbons atoms in the first intermediate stream, and hence lower percentages in the second intermediate stream will be required where the process stream to step (a) comprises relatively more hydrocarbons with 2 or more carbons atoms, such that the absolute amount of hydrocarbons with 2 or more carbons atoms which remain in the second intermediate stream is maintained at a suitably low level. Typically, the second intermediate stream comprises less than 1.0% by mole, of hydrocarbons with 2 or more carbons atoms.

Typically (i.e. using the temperatures required to achieve less than this percentage of hydrocarbons with two or more carbon atoms in the second intermediate stream) the first intermediate stream will comprise at least 2% of the carbon monoxide that was present in the initial process stream, and at least 10%, usually at least 20%, of the methane that was present in the initial process stream. The first intermediate stream will also usually comprise less than 1% of the hydrogen that was present in the initial process stream.

The first intermediate stream is subsequently treated to recover the methane, carbon monoxide and hydrogen therein. Thus, in step (b) of the process of the present invention the first intermediate stream is passed to a second separation step to separate the first intermediate stream into a product stream comprising hydrocarbons with 2 or more carbons atoms and a third intermediate stream comprising carbon monoxide, hydrogen and methane.

The second separation step may comprise vapour/liquid separation in a flash drum or in a rectification column.

Alternatively, the second separation step may comprise a combined rectification and chilling process, such as a dephlegmator, a distillation column with side condensers or an advanced heat integrated rectifier system, such as described in U.S. Pat. No. 6,343,487.

Preferably the second separation step comprises a distillation column that contains both a rectification section and a stripping section and which acts as a demethaniser.

Step (b) of the process of the present invention produces a product stream comprising hydrocarbons with 2 or more carbons atoms. As with step (a), the amount of separation required in step (b) will depend on the actual amount of hydrocarbons with 2 or more carbons atoms in the first intermediate stream, but, in general, the product stream comprising hydrocarbons with 2 or more carbons atoms typically comprises at least 90% by mole of the hydrocarbons with two or more carbon atoms that were present in the first intermediate stream, such as at least 95% by mole of the hydrocarbons with 2 or more carbons atoms, and less than 1% of the carbon monoxide, hydrogen and methane that was present in the first intermediate stream. The product stream comprising hydrocarbons with 2 or more carbons atoms generally comprises less than 0.1% by weight of hydrogen and carbon monoxide.

If a purified ethylene stream is to be produced from the product stream comprising hydrocarbons with 2 or more carbons atoms then it is preferred that the product stream comprising hydrocarbons with 2 or more carbons atoms comprises less than 10 ppm by mole of hydrogen, less than 5 ppm by mole of carbon monoxide and less than 1000 ppm by mole of methane.

Typically, the third intermediate stream comprises less than 1.0%, by mole, of hydrocarbons with 2 or more carbons atoms.

As noted previously, steps (a) and (b) of the process of the present invention provide a “two-stage” separation of hydrocarbons with 2 or more carbon atoms from the methane, carbon monoxide and hydrogen components of the mixed gas feed. Steps (a) and (b) may be performed at any suitable pressures. Typically, step (a) is performed at a higher pressure than step (b), and under these conditions the “two-stage” separation is significantly less energy intensive than a single stage process (which would generally need to be operated at a pressure similar to the higher pressure of step (a) to achieve effective separation).

Step (a) is preferably performed at a pressure in the range of 10-40 barg.

Step (b) is preferably performed at a pressure in the range 2-20 barg.

The second and third intermediate streams together comprise essentially all the carbon monoxide, hydrogen and methane that was present in the initial process stream (and generally less than 0.5% by moles of hydrocarbons with 2 or more carbons atoms).

In step (c) of the process of the present invention these streams are treated in a cryogenic separation system to separate the carbon monoxide, and optionally the hydrogen, therein. A purified methane stream may also be obtained.

In one embodiment the second intermediate stream is passed to a methane wash tower wherein said stream is contacted with methane to give an overheads stream comprising predominantly hydrogen (which may be used as a fuel, may be recovered as a hydrogen-containing product stream or may be sent to a hydrogen recovery process to further purify the hydrogen stream) and a bottoms stream comprising carbon monoxide and methane. The bottoms stream is then passed, with at least a portion of the third intermediate stream, to a carbon monoxide recovery process comprising a hydrogen stripper column and a carbon monoxide purification column.

Preferably both the bottoms stream and the third intermediate stream are passed to the hydrogen stripper column, with the third intermediate stream being used as a reflux liquid stream. A hydrogen-rich stream is recovered from the top of the hydrogen stripper and a bottoms stream rich in carbon monoxide and methane is passed to the carbon monoxide purification column where the carbon monoxide and methane are separated as respective carbon monoxide-rich and methane-rich product streams. The recovered hydrogen-rich stream from the top of the hydrogen stripper may be used as a fuel or sent to a hydrogen recovery process to produce a purified hydrogen stream (optionally after combination with the hydrogen-rich stream separated from the methane wash tower).

In a second embodiment the second and third intermediate streams are treated separately until they enter a common carbon monoxide purification column. This embodiment takes advantage of the fact that the two-step separation of hydrocarbons with two or more carbon atoms to produce the second and third intermediate streams also produces some separation of methane and carbon monoxide (the third intermediate stream has a higher methane to carbon monoxide ratio whereas the second intermediate stream has lower methane to carbon monoxide ratio compared to the initial process stream). In one example of this second embodiment, the second intermediate stream is passed to a rectification column where it is contacted with a reflux liquid stream comprising carbon monoxide which has itself been separated from the overhead stream from the rectification column. This is achieved by partially condensing the overheads stream from the rectification column comprising predominantly hydrogen and carbon monoxide (and little methane) to separate a hydrogen-rich stream (which may be used as a fuel or sent to a hydrogen recovery process to produce a purified hydrogen stream) and a carbon monoxide-rich stream, at least a portion of which can be used as the reflux liquid. The bottoms stream from the rectification column comprises predominantly methane and carbon monoxide, with some hydrogen and is passed to a hydrogen stripper column. A hydrogen-rich stream is recovered from the top of the hydrogen stripper and a bottoms stream rich in carbon monoxide and methane is passed to a carbon monoxide purification column wherein the carbon monoxide and methane are separated as respective carbon monoxide-rich and methane-rich product streams.

The third intermediate stream is also treated to remove hydrogen therein, preferably in a flash drum, to produce a hydrogen-rich stream and a stream rich in carbon monoxide and methane. The hydrogen-rich stream may be used as a fuel or sent to a hydrogen recovery process to produce a purified hydrogen stream (optionally after combination with the hydrogen-rich stream separated from the treatment of the second intermediate stream). The stream rich in carbon monoxide and methane is passed to a carbon monoxide purification column wherein the carbon monoxide and methane are separated as respective carbon monoxide-rich and methane-rich product streams.

The hydrocarbons with 2 or more carbons atoms are typically present in the process stream in a range 2 to 30 mol %, preferably 2 to 20 mol %.

Typical concentrations of the carbon monoxide, hydrogen and methane are:

| | |
|---|---|
| carbon monoxide | 2 to 50 mol %, such as 5 to 40 mol %; |
| hydrogen: | 5 to 80 mol %, such as 20 to 70 mol %; and |
| methane | 5 to 60 mol %, such as 10 to 40 mol %. |

In a preferred embodiment of the present invention, the process stream comprising carbon monoxide, hydrogen, methane and hydrocarbons with 2 or more carbons atoms is derived from the product stream of an autothermal cracking process for the production of olefins.

Autothermal cracking is a route to olefins in which a hydrocarbon-containing feedstock is mixed with oxygen and passed over an autothermal cracking catalyst. The autothermal cracking catalyst is capable of supporting combustion beyond the fuel rich limit of flammability. Combustion is initiated on the catalyst surface and the heat required to raise the reactants to the process temperature and to carry out the endothermic cracking process is generated in situ. Generally the hydrocarbon-containing feedstock and the oxygen are passed over a supported catalyst capable of supporting combustion beyond the fuel rich limit of flammability to produce the olefin product. The autothermal cracking process is described in EP 332289B; EP-529793B; EP-A-0709446 and WO 00/14035.

The catalyst capable of supporting combustion beyond the fuel rich limit of flammability usually comprises a Group VIII metal as its catalytic component. Suitable Group VIII metals include platinum, palladium, ruthenium, rhodium, osmium and iridium. Rhodium, and more particularly, platinum and palladium are preferred. Typical Group VIII metal loadings range from 0.01 to 100 wt %, preferably, between 0.01 to 20 wt %, and more preferably, from 0.01 to 10 wt % based on the total dry weight of the catalyst.

Where a Group VIII catalyst is employed, it is preferably employed in combination with a catalyst promoter. The promoter may be a Group IIIA, IVA, and/or VA metal. Alternatively, the promoter may be a transition metal; the transition metal promoter being a different metal to that which may be employed as the Group VIII transition metal catalytic component.

Autothermal cracking may be used to convert both liquid and gaseous hydrocarbons into olefins. Suitable liquid hydrocarbons include naphtha, gas oils, vacuum gas oils and mixtures thereof. Preferably, however, gaseous hydrocarbons such as ethane, propane, butane and mixtures thereof are employed.

The hydrocarbon-containing feedstock may be fed with any suitable oxygen-containing gas. Suitably, the oxygen-containing gas is molecular oxygen, air and/or mixtures thereof. The oxygen-containing gas may be mixed with an inert gas such as nitrogen or argon.

Preferably the hydrocarbon-containing feedstock and oxygen-containing gas are fed to the autothermal cracker at a ratio of hydrocarbon to oxygen-containing gas of 5 to 16 times, preferably 5 to 13.5 times, more preferably 6 to 10 times, the stoichiometric ratio of hydrocarbon to oxygen-containing gas required for complete combustion of the hydrocarbon to carbon dioxide and water.

The hydrocarbon is passed over the catalyst at a gas hourly space velocity of greater than 10,000 h⁻¹, preferably above 20,000 h⁻¹ and most preferably, greater than 100,000 h⁻¹. It will be understood, however, that the optimum gas hourly space velocity will depend upon the pressure and nature of the feed composition.

Additional feed components may be co-fed into the autothermal cracker, such as hydrogen, carbon monoxide, carbon dioxide or steam. Preferably, hydrogen is co-fed with the hydrocarbon-containing feedstock and oxygen-containing gas into the autothermal cracker. Suitably, the molar ratio of hydrogen to oxygen-containing gas is in the range 0.2 to 4.

Hydrogen co-feeds are advantageous because, in the presence of the catalyst, the hydrogen combusts preferentially relative to the hydrocarbon, thereby increasing the olefin selectivity of the overall process.

The autothermal cracking process is suitably be carried out at a catalyst exit temperature in the range 600° C. to 1200° C., preferably, in the range 850° C. to 1050° C. and, most preferably, in the range 900° C. to 1000° C. To avoid further reactions taking place, the ATC product stream should be rapidly cooled, typically by cooling to between 750-600° C. within 20 milliseconds of formation. Advantageously wherein the autothermal cracking process is operated at a pressure of greater than 20 barg the products are cooled to between 750-600° C. within 10 milliseconds of formation.

The reaction products are quenched with water as they emerge from the autothermal cracker, typically in a suitable quench tower. The ATC product stream, in addition to olefins, typically comprises unreacted hydrocarbons, hydrogen, carbon monoxide, methane, and small amounts of acetylenes, aromatics and carbon dioxide, which need to be separated from the desired olefins.

Typically, carbon dioxide is removed from the ATC product stream first, for example, using an amine-based absorption system such as MEA or TEA (or mixtures of both), or other commercially available CO₂ removal process.

Any residual water may be removed next. Any suitable drying process may be used, for example by use of a suitable molecular sieve.

All or a portion of this ATC product stream (after carbon dioxide removal and after water removal) may then be passed as a process stream comprising carbon monoxide, hydrogen, methane and hydrocarbons with 2 or more carbons atoms, for treatment to remove carbon dioxide, and optionally hydrogen, as in the first aspect of the present invention.

In one preferred embodiment, the ATC product stream (or a portion thereof), after treatment to remove carbon dioxide and/or water, can be passed to a chilling train comprising one or more stages in which chilling and partial condensation occur prior to being passed as a process stream for treatment as in the first aspect of the present invention.

Within each stage of the chilling train, the chilling and partial condensation are followed by liquid and vapour separation. The vapour is passed to the next chilling/partial condensation stage (if any), the vapour stream from the final chilling/partial condensation stage forming a stream comprising carbon monoxide, hydrogen, methane and hydrocarbons with 2 or more carbons atoms that may be passed as a process stream for treatment as in the first aspect of the present invention. The separated liquid stream or streams from the one or more stages of the chilling train may be combined and processed separately, for example, as an additional feed to the second separation step of step (b) of the first aspect of the present invention.

In some embodiments it may be desirable to include a front-end rectification column after the treatment to remove carbon dioxide and/or water and prior to the chilling train comprising one or more stages in which chilling and partially condensation occur described above. Front-end rectification may be desired where the ATC product stream comprises significant quantities of hydrocarbons having 2 or more carbon atoms and it is desired to remove some of the heavier of these hydrocarbons. For example, it may be desired to remove C3 and heavier or C4 and heavier hydrocarbons from the ATC product stream through the use of a front-end deethanizer or front-end depropanizer, respectively. Generally, the ATC product stream (or a portion thereof), after treatment to remove carbon dioxide and/or water, may be chilled and passed to the front-end rectification column, wherein the heavier components, for example the C3 and heavier or the C4 and heavier hydrocarbons, would be removed as a bottoms stream. The bottoms stream can be separately treated as required.

The remaining components of the ATC product stream would be recovered as the overhead stream from the front-end rectification column and this overhead stream, containing a majority of the hydrogen and CO that enters the front-end rectification column, would then enter the chilling train and then be passed as a process stream for treatment as in the first aspect of the present invention, as described above.

The ATC product stream (or a portion thereof) may need to be compressed before the at least a portion of it is passed as the process stream comprising carbon monoxide, hydrogen, methane and hydrocarbons with 2 or more carbons atoms to the process of the first aspect of the present invention. Compression may be performed in a suitable compressor at any convenient stage.

The present invention will now be illustrated with respect to the Figure and the example, wherein

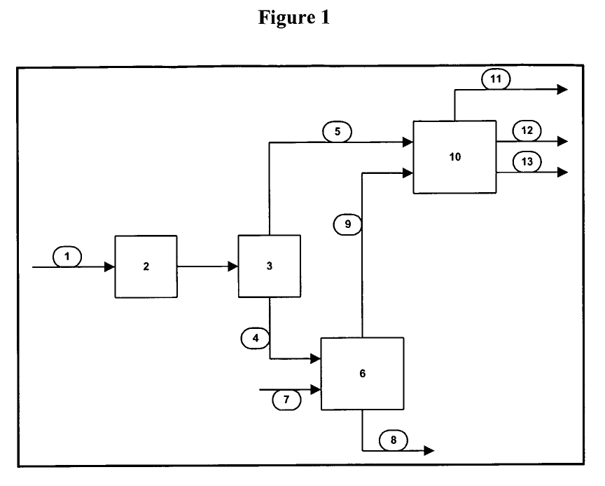

FIG. 1 represents, in schematic form, a preferred configuration for the separation of carbon monoxide from a process stream comprising carbon monoxide, hydrogen, methane and hydrocarbons with 2 or more carbons atoms,

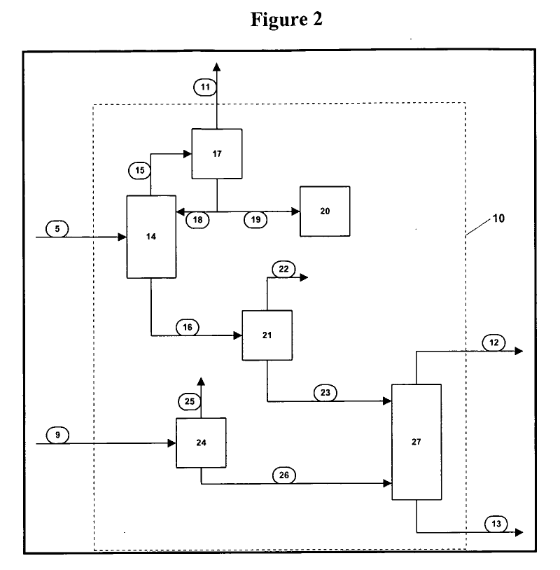

FIG. 2 represents, in schematic form, a preferred configuration for the cryogenic separation step (c) to produce product streams comprising hydrogen, methane and carbon monoxide,

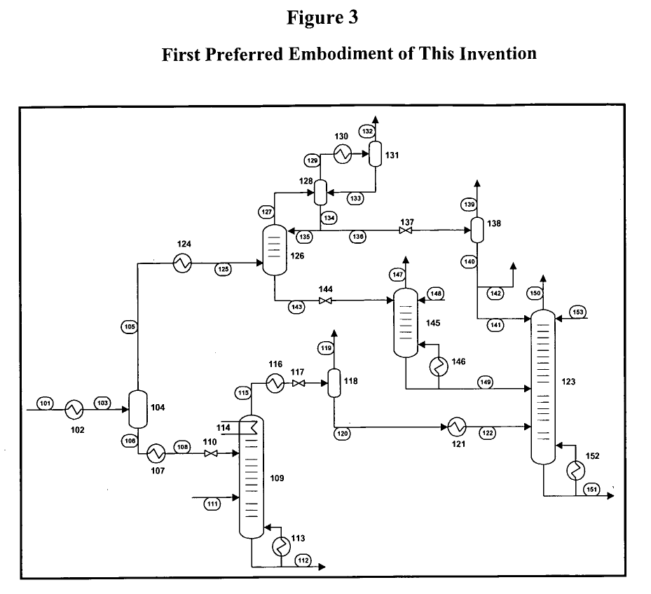

FIG. 3 represents a more detailed first embodiment of this invention,

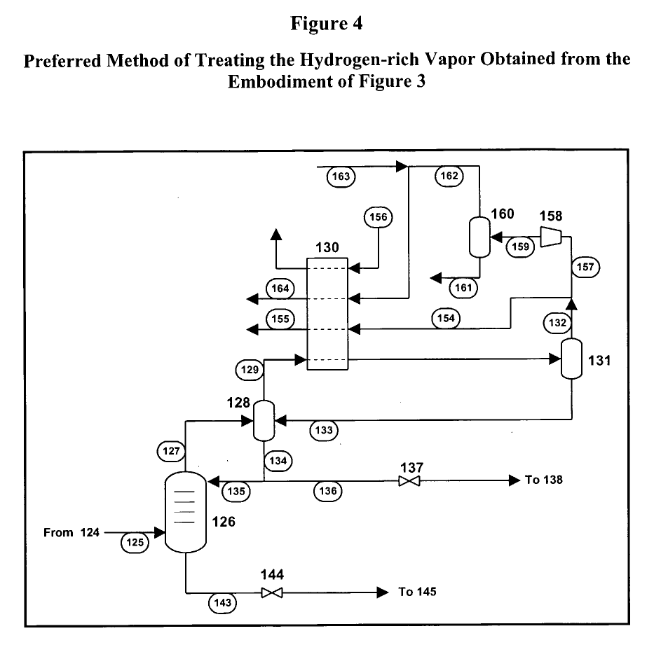

FIG. 4 represents a preferred method of treating the hydrogen-rich vapor obtained from the embodiment of FIG. 3, and

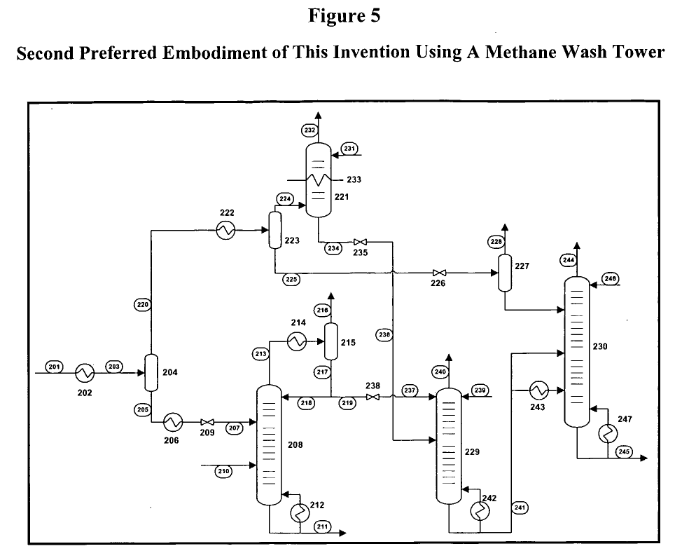

FIG. 5 represents a second embodiment of the process of this invention, one which utilizes a methane wash column.

In FIG. 1 a process stream (1) containing primarily hydrogen, carbon monoxide, methane, and heavier hydrocarbons is passed through an exchanger (2) wherein it is chilled and partially condensed before being passed to a first separation step (3) which is a drum in which liquid and vapour phases are separated as respective first and second intermediate streams (4) and (5). The first intermediate stream (4) comprises hydrocarbons with 2 or more carbon atoms and a reduced quantity of carbon monoxide, hydrogen and methane, and is passed to a second separation step (6), which is a distillation column that contains both a rectification section and a stripping section and acts as a demethanizer. There may also be passed to the second separation step (6) a further liquid stream (7) comprising hydrocarbons from other parts of the overall process, such as from liquid streams from one or more stages of a chilling train to which an ATC product stream has been passed prior to production of the process stream (1).

This second separation step (6) produces a product stream comprising hydrocarbons with 2 or more carbon atoms (8) and a third intermediate stream comprising carbon, monoxide, hydrogen and methane, which is passed to a cryogenic separation section, 10, along with the second intermediate stream (5), wherein are produced a purified hydrogen stream (11), a purified carbon monoxide stream (12) and a purified methane stream (13).

With reference to FIG. 2, the second intermediate stream (5) is cooled (not shown) and passed to a rectification column (14) wherein it is contacted with a counterflowing, downflowing reflux liquid stream to produce an overhead vapour stream (15) rich in hydrogen and carbon monoxide and a liquid bottoms stream (16) comprising predominantly methane and carbon monoxide, with some hydrogen. The reflux liquid stream is itself produced from condensation of the overhead vapour (15) in a separation step (17) which produces a purified hydrogen stream (11) and a carbon monoxide rich stream at least a part of which (18) is recycled as the reflux liquid. The remaining portion of the carbon monoxide rich stream (19) may be treated (20), for example to separate hydrogen therefrom (detail not shown).

The liquid bottoms stream (16) from the rectification column (14) is passed to a hydrogen stripper column (21) to produce an overhead product stream (22) comprising predominantly hydrogen and a bottoms liquid (23) comprising methane and carbon monoxide. Stripping vapor may be generated by passing a portion of the bottoms liquid (23) back to the hydrogen stripper column via a reboiler (not shown). If desired a methane-rich liquid (not shown) can be used as a reflux liquid in the hydrogen stripper column (21).

The third intermediate stream (9) is partially condensed and flashed to a lower pressure (not shown), before passing to a separation drum (24), to produce an overhead vapour stream (25) comprising primarily hydrogen, with smaller amounts of methane and carbon monoxide, and a bottoms liquid stream (26) comprising predominantly methane and carbon monoxide.

Streams (23) and (26) are both passed to separate points on a carbon monoxide purification tower (27), wherein a purified carbon monoxide stream (12) and a purified methane stream (13) are produced.

The embodiment of FIG. 2 produces a number of hydrogen-rich vent streams ((22), (25) and optionally any hydrogen recovered from stream (19)) which comprise predominantly hydrogen, but also some carbon monoxide and methane. If desired one or all of these streams can be chilled and partially condensed to provide a carbon monoxide-containing liquid, and if required the carbon monoxide-containing liquid can be directed to the carbon monoxide purification column (27) for further purification.

FIG. 3 contains an illustrative depiction of a first embodiment of this invention. All major separation, heating, and cooling steps have been shown. Some details of the process design that are well know to those skilled in the art, such as some vapor-liquid separation drums, process control valves, pumps and the like have been omitted from the drawing in order to demonstrate more clearly the key concepts of the invention. It should be noted that for clarity all heat exchangers in this detailed description are shown as individual exchangers. In practice they would preferably be combined into one or more multi-pass cryogenic heat exchangers, as is well known to those skilled in the art.

In FIG. 3 a pressurized mixed hydrocarbon stream containing primarily hydrogen, carbon monoxide, methane, and heavier hydrocarbons enters the process as Stream 101. It is chilled and partially condensed in exchanger 102 to form stream 103. Stream 103 enters drum 104 in which the vapor and liquid are separated. The overhead vapor stream 105 is relatively enriched in CO and relatively depleted in hydrocarbons heavier than methane. The liquid stream 106 is relatively enriched in hydrocarbons heavier than methane and relatively depleted in CO, although stream 106 does contain significant amounts of CO that can be economically recovered.

It is clear to those skilled in the art that many design options exist for the separation of stream 101 into a stream relatively enriched in CO and a stream or streams relatively depleted in CO. In FIG. 3 this separation is accomplished with a simple partial condensation and separation drum. Alternatively, this separation could also operate by rectification, or could combine rectification and chilling operations. Such combined chilling and rectification options would include, but not be limited to, a dephlegmator, a distillation column with side condensers, or the advanced heat integrated rectification designs of U.S. Pat. No. 6,343,487 and U.S. Pat. No. 4,496,381, among others.

Stream 106 would typically contain carbon monoxide, methane, heavier hydrocarbons, and some dissolved hydrogen. It can be optionally heated or cooled by exchanger 107 before being sent as stream 108 to a second separation unit 109. In principle 109 could be a simple flash drum in which the vapor and liquid of stream 108 are separated. Alternatively it could operate by rectification or it could combine rectification and chilling operations, such as a dephlegmator or a distillation column with side condensers, among others.

In FIG. 3 separation unit 109 is a distillation column that contains both a rectification section and a stripping section and acts as a demethanizer. If the pressure of 109 is significantly lower than the pressure of 104, then a throttling means can be used such as valve 110 shown. Stream 108 enters at an intermediate point on the demethanizer column 109, somewhere between the top and bottom contacting trays. Other feeds from other parts of the process may also enter 109, as indicated by stream 111. The bottoms stream 112 is a product stream comprising hydrocarbons with 2 or more carbon atoms and little if any methane or lighter components. Stripping vapor is provided to 109 with reboiler 113. 109 is condensed with partial condenser 114, shown in FIG. 3 as an internal condenser.

The overhead stream 115 is partially condensed in 116, the partially condensed stream is flashed to a lower pressure through valve 117, and the vapor and liquid in the resulting flashed stream are separated in drum 118. The vapor stream 119 contains primarily hydrogen along with some methane and carbon monoxide. It can be sent to a hydrogen recovery process or warmed and used as fuel. The liquid stream 120 is warmed and partially vaporized in 121 and then directed as stream 122 to the CO purification column 123. Other ways of treating stream 120 could be envisioned, such as splitting it and warming only a fraction of the stream before entering 123. Such options are within the scope of this invention.

Stream 105, the overhead vapor of drum 104, is chilled and partially condensed in 124 to form stream 125. Stream 125 enters the bottom of the CO rectification column 126. Alternatively, the vapor and liquid could be separated and only the vapor fraction of stream 125 sent to 126. In 126 the upflowing vapor is contacted with downflowing reflux liquid, thus separating CO and methane such that the gross overhead vapor stream 127 contains primarily hydrogen and CO and little methane. Stream 127 is directed to reflux recontacting drum 128. The overhead vapor stream 129 is chilled and partially condensed in 130. The vapor and liquid in the resulting stream are separated in reflux drum 131. Stream 132 is the vapor from 131 and constitutes the net overhead vapor of the CO rectification column 126. Stream 132 consists primarily of hydrogen with a small amount of carbon monoxide. It can be recovered as a purified hydrogen product, sent to further purification steps, or warmed and used as fuel.

Liquid stream 133 is directed to the reflux recontacting drum 128. There it meets and contacts the vapor stream 127. This reflux recontacting serves to chill the overhead vapor stream of 128 while warming the remaining liquid. The warmed liquid stream 134 is split into two streams. The first, stream 135, is directed back to the top of 126 as reflux liquid and the other is withdrawn as stream 136. It will be apparent to those skilled in the art that in practice drum 128 and column 126 can be combined into a single vessel, and also that there may be benefit to having some vapor/liquid contacting stages within 128. In such a case stream 136 would be a liquid product drawn off of the first tray, or one of the top few trays, of 126. The two vessels are depicted in FIG. 3 as separate vessels to more easily show the concept of the reflux recontacting operation.

Stream 136 is flashed across valve 137 and the vapor and liquid in the resulting flashed stream are separated in drum 138. Vapor stream 139 contains primarily hydrogen along with some carbon monoxide. It can be sent to a hydrogen recovery process or warmed and used as fuel. The liquid from 138 is stream 140 and at least a portion of it is directed as reflux liquid stream 141 to the top of 123. If there is more liquid in stream 140 than is needed as reflux to 123, a liquid CO stream 142 may be recovered. This stream would typically be vaporized and reheated to recover refrigeration value, and then be compressed as part of the final CO product.

The bottoms stream 143 from column 126 contains primarily carbon monoxide, methane, and some dissolved hydrogen. It is flashed across valve 144 and enters as feed to the top of the hydrogen stripper column 145. Column 145 serves to remove dissolved hydrogen from stream 143. Stripping vapor is generated with reboiler 146. The overhead product stream 147 contains primarily hydrogen and can be sent to a hydrogen recovery process, or warmed and used as fuel. If desired a methane-rich liquid, shown as stream 148 in FIG. 3, can be directed as reflux to the top of 145, as has been disclosed in the art. The stripped bottoms liquid stream 149 contains primarily carbon monoxide and methane. It is directed to the CO purification column 123 and enters at a location above that of feed stream 122 and below that of stream 141. Other ways of treating stream 149 could be envisioned, such as warming or chilling all or a portion of the stream before it enters 123.

The CO purification tower produces a purified CO product as the overhead stream 150 and a purified methane bottoms stream 151. Stripping vapor is supplied to the bottom of the tower with reboiler 152, and if needed additional reflux liquid can be supplied through stream 153, either through partial condensation of stream 150 or as part of a CO refrigeration system.

The embodiment of FIG. 3 produces a number of hydrogen-rich vent streams, in particular streams 119, 139, and 147. These streams contain primarily hydrogen, but also some methane and/or CO. If desired one or all of these streams can be chilled and partially condensed to provide a CO-containing liquid, and the CO-containing liquid directed to column 123 for further purification if required.

FIG. 4 depicts a preferred method of treating the hydrogen-rich vapor obtained from the embodiment of FIG. 3. This method produces both a high-pressure hydrogen stream and a low-pressure hydrogen stream. The high-pressure hydrogen stream could be sold as product, recycled to an olefin-producing step, for example an autothermal cracking step, or directed to other suitable uses. The low-pressure hydrogen stream could be used as plant fuel.

FIG. 4 contains some streams and process equipment that appear in FIG. 3. The stream and process unit numbers for these common elements are the same in FIGS. 3 and 4. The exchanger 130, shown as a simple exchanger in FIG. 3, is shown in FIG. 4 as a multi-pass exchanger to more clearly depict the method of FIG. 4. In the method of FIG. 4 stream 132 is split into two streams. Stream 154 constitutes the high-pressure hydrogen stream. It is re-warmed in 130 to recover refrigeration value. The warmed high-pressure hydrogen product stream 155 can be further warmed elsewhere in the process and recovered as a purified hydrogen product. An external refrigerant stream 156 can also be directed to 130 if additional refrigeration is needed in this exchanger.

The other portion of stream 132, stream 157, is directed to an expander 158. The expander reduces the pressure of stream 157, thereby chilling and partially condensing it. Alternate means for reducing the pressure of the stream could also be employed, such as a simple valve. The lower-pressure, partially condensed stream 159 is directed to drum 160 where the vapor and liquid are separated. The liquid stream 161 is enriched in carbon monoxide and is recovered as purified CO product. It can, for example, be directed to the low-pressure side of a refrigeration cycle that utilizes purified carbon monoxide as the working fluid. Such refrigeration cycles are well known to those skilled in the art. The vapor stream 162 consists primarily of hydrogen and constitutes the low-pressure hydrogen stream. It is can optionally be combined with one or more other hydrogen vent streams, designated as single stream 163 in FIG. 4, and warmed in exchanger 130 to produce the warmed low-pressure hydrogen stream 164. This stream can be further warmed elsewhere in the process and can be used as fuel or as a purified hydrogen product.

The concept of this method can be modified as desired to provide more than two hydrogen streams, or to produce the hydrogen streams in varying amounts and final pressures.

FIG. 5 depicts a second embodiment of the process of this invention, one that utilizes a methane wash column. A pressurized mixed hydrocarbon stream containing primarily hydrogen, carbon monoxide, methane, and heavier hydrocarbons enters the process as stream 201. It is chilled and partially condensed in exchanger 202 to form stream 203. Stream 203 enters drum 204. In principle 204 can be a simple flash drum as shown or alternatively it could operate by rectification, in which upflowing vapor is contacted with a downflowing liquid. Additionally, 204 could combine rectification and chilling operations, such as a dephlegmator, a distillation column with side condensers, or the advanced heat integrated rectification designs of U.S. Pat. No. 6,343,487 and U.S. Pat. No. 4,496,381, among others.

Liquid stream 205 contains carbon monoxide, methane, heavier hydrocarbons, and some dissolved hydrogen. It can be optionally heated or cooled by exchanger 206 before being sent as stream 207 to a second separation unit 208. In principle 208 could be a simple flash drum in which the vapor and liquid of stream 208 are separated. Alternatively it could operate by rectification, in which upflowing vapor is contacted with a downflowing liquid. Additionally it could combine rectification and chilling operations, such as a dephlegmator or a distillation column with side condensers, among others.

In FIG. 5 separation unit 208 is a distillation column that contains both a rectification section and a stripping section and acts as a demethanizer. If the pressure of 208 is significantly lower than the pressure of 204, then a throttling means can be used such as valve 209 shown. Stream 207 enters at an intermediate point on the demethanizer column 208, somewhere between the top and bottom contacting tray. Other feeds from other parts of the process may also enter 208, as indicated by stream 210 in FIG. 5. The bottoms stream 211 constitutes a product stream comprising hydrocarbons with 2 or more carbon atoms and little if any methane or lighter components. Stripping vapor is provided to 208 with reboiler 212. The overhead stream 213 is partially condensed in 214 and the vapor and liquid are separated in drum 215. The vapor stream 216 contains primarily hydrogen along with some methane and carbon monoxide. It can be sent to a hydrogen recovery process or warmed and used as fuel. The liquid stream 217 is split into two streams. A portion, stream 218, is directed back to column 208 as reflux. The remainder, stream 219, is directed to the CO recovery process.

Stream 220, the vapor from 204, is sent to the methane wash column 221. It may first be chilled and partially condensed as shown in exchanger 222. In this case the vapor and liquid can be separated in drum 223 if desired. The vapor stream 224 enters the bottom of the methane wash tower 221. The liquid stream 225 is expanded through valve 226 and the resulting vapor and liquid are separated in drum 227. The vapor stream 228 is rich in hydrogen and can be sent to a hydrogen recovery process or warmed and used as fuel. It could also be sent to the hydrogen stripper column 229 as has been previously disclosed (U.S. Pat. No. 6,269,657). The liquid stream from 227 enters the CO purification column 230.

A chilled methane-rich wash liquid stream 231 enters the top of the methane wash tower 221 and countercurrently contacts the upflowing vapor from stream 224. The majority of the methane and carbon monoxide are dissolved in the downflowing liquid, so that the overhead vapor stream 232 is a purified hydrogen product. An intermediate cooler or coolers may be used in the methane wash column, shown in FIG. 5 as exchanger 233. The purpose of this intermediate cooler is to remove some of the heat of condensation and heat of solution of the methane and carbon monoxide that are being condensed in column 221 and thereby reduce the amount of liquid methane adsorbent 231 required to achieve the desired level of CO recovery to the absorber bottoms stream 234.

The bottoms stream 234 is reduced in pressure across valve 235 and then enters the hydrogen stripper column 229 as stream 236. Stream 219 enters the top of 229 as reflux liquid stream 237 after being throttled in valve 238. Optionally, other methane-rich reflux liquid can be directed to the top of 229, depicted in FIG. 5 as stream 239. The purpose of reflux liquid to 229 is to reduce the amount of carbon monoxide that is lost to the overhead stream 240. The overhead liquid stream 219 from the demethanizer column has been found to be particularly well suited for use as reflux liquid to 229.

The overhead stream 240 contains primarily hydrogen and can be sent to a hydrogen recovery process or warmed and used as fuel. The bottoms stream 241 contains primarily carbon monoxide and methane and little if any hydrogen or heavier hydrocarbons. Stripping vapor is provided to the bottom of 229 by reboiler 242. Stream 241 is directed to the CO purification column 230. If desired, the stream may be split into two or more streams and partially heated in 243 as shown.

The CO purification column produces a purified carbon monoxide product as the overhead stream 244 and a purified liquid methane product as the bottoms stream 245. Reflux liquid is provided to the column via stream 246, either by condensation of stream 244 or as part of a CO refrigeration system. Stripping vapor is generated in reboiler 247. As is well known to those skilled in the art, a fraction of the methane-enriched liquid product in stream 245 may be chilled and used as the methane-rich liquids of streams 239 and/or 231.

### EXAMPLE 1

A CO recovery based on the embodiment of FIG. 3 was simulated using commercially available process simulation software. The mixed hydrocarbon feed contains primarily hydrogen, carbon monoxide, ethylene, and ethane and is derived from the effluent of an autothermal cracking reactor. The composition of key streams is given in Table 1. Stream 111 in Table 1 is the sum of three separate liquid streams that enter column 109. These three streams range in temperature from −40 to −100° C. and are derived from the progressive chilling and partial condensation of the autothermal cracking reactor effluent. The individual heat exchanger duties are given in Table 2. In all cases the stream and equipment numbers relate to those shown in FIG. 3.

It is to be noted that while the first separation step alone (104) produces a first intermediate stream (stream 106) having the majority of the hydrocarbons having 2 or more carbon atoms, this stream also comprises significant quantities of methane and carbon monoxide. In contrast, the two-stage process of this invention provides improved recovery of carbon monoxide (and methane), and produces separate product streams of high purity.

Particularly, it can be seen that the two-stage removal of heavy hydrocarbons in the process of this invention allows for a very high recovery of both carbon monoxide and hydrocarbons heavier than methane to the purified product streams. The recovery of CO to product streams 142 and 150 is 90.2%. Additional CO recovery to stream 161 according the method of FIG. 4 increases overall CO recovery to 92.6%. The recovery of hydrocarbons heavier than methane to product stream 112 is 99.8%. The simultaneous high recovery of both CO and hydrocarbons heavier than methane to their final product streams is a key benefit of this invention not provided by the prior art. In addition, significant energy is saved by operating the second separation step (109) at a lower pressure than the first separation step (and compared to a single stage process attempting to achieve improved recovery).

It is to be further noted that the two-stage removal of heavy hydrocarbons in this process accomplishes a degree of pre-separation of the carbon monoxide and methane that is present in the feed stream. This can be clearly seen by comparing the compositions of stream 101, stream 105, and stream 115 in Table 1. The molar ratio of carbon monoxide to methane is significantly higher in stream 105 (1.79) than in stream 115 (0.26). Through the method of this invention this pre-separation of carbon monoxide and methane is used to advantage by directing them, after some intermediate processing steps, to different locations on the CO purification tower 123.

TABLE 1
Flows and Conditions for Streams of Example (FIG. 3)

| Stream No. | 100 | 105 | 106 | 111 | 112 | 115 | 119 | 122 | 125 | 127 | 129 |
|---|---|---|---|---|---|---|---|---|---|---|---|
| Temperature (Deg C.) | −130.0 | −141.0 | −141.0 | −40 to −110 | −43.4 | −131.4 | −172.6 | −139.8 | −172.0 | −177.2 | −178.3 |
| Pressure (barg) | 24 | 24 | 24 | 25 | 9 | 8 | 6 | 5 | 24 | 24 | 24 |
| Vapor Fraction | 0.96 | 1.00 | 0.00 | 0.00 | 0.00 | 1.00 | 1.00 | 0.54 | 0.76 | 1.00 | 1.00 |

Molar flows (kmol/hr)

| | | | | | | | | | | | |
|---|---|---|---|---|---|---|---|---|---|---|---|
| Hydrogen | 3674.9 | 3668.6 | 6.3 | 78.6 | 0.0 | 84.9 | 79.8 | 5.0 | 3668.6 | 3643.7 | 3635.9 |
| CO | 1472.8 | 1412.9 | 59.9 | 199.4 | 0.0 | 259.3 | 29.0 | 230.3 | 1412.9 | 1036.1 | 965.9 |
| Methane | 1065.2 | 790.0 | 275.2 | 726.7 | 1.5 | 1000.4 | 5.8 | 994.6 | 790.0 | 4.9 | 1.0 |
| Ethylene | 141.2 | 9.4 | 131.9 | 3527.1 | 3656.2 | 2.7 | 0.0 | 2.7 | 9.4 | 0.0 | 0.0 |
| Ethane | 32.1 | 0.6 | 31.4 | 3170.8 | 3202.2 | 0.0 | 0.0 | 0.0 | 0.6 | 0.0 | 0.0 |
| Acetylene | 0.5 | 0.0 | 0.4 | 33.5 | 34.0 | 0.0 | 0.0 | 0.0 | 0.0 | 0.0 | 0.0 |
| Propylene | 0.0 | 0.0 | 0.0 | 231.0 | 231.0 | 0.0 | 0.0 | 0.0 | 0.0 | 0.0 | 0.0 |
| Propane | 0.0 | 0.0 | 0.0 | 50.1 | 50.1 | 0.0 | 0.0 | 0.0 | 0.0 | 0.0 | 0.0 |
| MAPD | 0.0 | 0.0 | 0.0 | 5.6 | 5.6 | 0.0 | 0.0 | 0.0 | 0.0 | 0.0 | 0.0 |
| C4+ | 0.0 | 0.0 | 0.0 | 252.9 | 252.9 | 0.0 | 0.0 | 0.0 | 0.0 | 0.0 | 0.0 |

| Stream No. | 132 | 133 | 134 | 136 | 139 | 140 | 142 | 143 | 147 | 149 | 150 | 151 |
|---|---|---|---|---|---|---|---|---|---|---|---|---|
| Temperature (Deg C.) | −190.5 | −190.5 | −178.3 | −178.3 | −180.3 | −180.3 | −180.3 | −172.1 | −172.3 | −157.5 | −171.0 | −134.9 |
| Pressure (barg) | 24 | 24 | 24 | 24 | 6 | 6 | 6 | 24 | 8 | 8 | 5 | 5 |
| Vapor Fraction | 1.00 | 0.00 | 0.00 | 0.00 | 1.00 | 0.00 | 0.00 | 0.00 | 1.00 | 0.00 | 1.00 | 0.00 |

Molar flows (kmol/hr)

| | | | | | | | | | | | | |
|---|---|---|---|---|---|---|---|---|---|---|---|---|
| Hydrogen | 3611.1 | 24.8 | 14.7 | 17.9 | 15.3 | 2.6 | 0.6 | 39.6 | 38.2 | 1.4 | 8.8 | 0.0 |
| CO | 290.9 | 674.9 | 335.4 | 409.8 | 15.7 | 394.1 | 88.2 | 712.2 | 25.1 | 687.1 | 1239.9 | 25.3 |
| Methane | 0.0 | 0.9 | 2.2 | 2.7 | 0.0 | 2.7 | 0.6 | 787.3 | 2.0 | 785.2 | 3.6 | 1785.8 |
| Ethylene | 0.0 | 0.0 | 0.0 | 0.0 | 0.0 | 0.0 | 0.0 | 9.4 | 0.0 | 9.4 | 0.0 | 12.1 |
| Ethane | 0.0 | 0.0 | 0.0 | 0.0 | 0.0 | 0.0 | 0.0 | 0.6 | 0.0 | 0.6 | 0.0 | 0.6 |
| Acetylene | 0.0 | 0.0 | 0.0 | 0.0 | 0.0 | 0.0 | 0.0 | 0.0 | 0.0 | 0.0 | 0.0 | 0.0 |
| Propylene | 0.0 | 0.0 | 0.0 | 0.0 | 0.0 | 0.0 | 0.0 | 0.0 | 0.0 | 0.0 | 0.0 | 0.0 |
| Propane | 0.0 | 0.0 | 0.0 | 0.0 | 0.0 | 0.0 | 0.0 | 0.0 | 0.0 | 0.0 | 0.0 | 0.0 |
| MAPD | 0.0 | 0.0 | 0.0 | 0.0 | 0.0 | 0.0 | 0.0 | 0.0 | 0.0 | 0.0 | 0.0 | 0.0 |
| C4+ | 0.0 | 0.0 | 0.0 | 0.0 | 0.0 | 0.0 | 0.0 | 0.0 | 0.0 | 0.0 | 0.0 | 0.0 |

TABLE 2
Heat Exchanger Duties

| Exchanger | Net Duty (kW) |
|---|---|
| 102 | −1139 |
| 107 | Not Used |
| 114 | −331 |
| 113 | 5567 |
| 116 | −3105 |
| 121 | 1840 |
| 124 | −3931 |
| 130 | −1446 |
| 146 | 380 |
| 152 | 730 |

1-15. (canceled)

16. A process for the separation of carbon monoxide and optionally hydrogen from a process stream comprising carbon monoxide, hydrogen, methane and 2-30 mole % hydrocarbons with 2 or more carbons atoms, said process comprising:
a) passing the process stream to a first separation step to separate the process stream into a first intermediate stream comprising at least 90% by mole of the hydrocarbons with 2 or more carbons atoms that were present in the process stream and a reduced quantity of carbon monoxide, hydrogen and methane, and a second intermediate stream comprising carbon monoxide, hydrogen and methane and a reduced quantity of hydrocarbons with 2 or more carbons atoms,
b) passing the first intermediate stream to a second separation step to separate the first intermediate stream into a product stream comprising at least 90% by mole of the hydrocarbons with 2 or more carbons atoms that were present in the first intermediate stream and less than 1% of the carbon monoxide, hydrogen and methane that was present in the first intermediate stream, and a third intermediate stream comprising carbon monoxide, hydrogen and methane, and
c) treating both the second and third intermediate streams in a cryogenic separation system to separate the carbon monoxide, and optionally the hydrogen, therein.

17. A process as claimed in claim 16, wherein the process produces a purified carbon monoxide stream, a purified hydrogen stream, a purified methane stream, and a product stream comprising hydrocarbons with 2 or more carbon atoms.

18. A process as claimed in claim 16, wherein the first separation step comprises partial condensation of the process stream, followed by vapor/liquid separation in a flash drum or in a rectification column.

19. A process as claimed in claim 16, wherein the first separation step comprises a combined rectification and chilling process, such as a dephlegmator, a distillation column with side condensers or an advanced heat integrated rectifier system.

20. A process as claimed in claim 16, wherein the first separation step produces a second intermediate stream comprising less than 1.0%, by mole, of hydrocarbons with 2 or more carbons atoms.

21. A process as claimed in claim 16, wherein the second separation step comprises vapor/liquid separation in a flash drum or in a distillation column.

22. A process as claimed in claim 16, wherein the second separation step comprises a combined rectification and chilling process, such as a dephlegmator, a distillation column with side condensers or an advanced heat integrated rectifier system.

23. A process as claimed in claim 16, wherein the second separation step comprises a distillation column that contains both a rectification section and a stripping section.

24. A process as claimed in claim 16, wherein the second separation step produces a third intermediate stream comprising less than 1.0%, by mole, of hydrocarbons with 2 or more carbons atoms.

25. A process as claimed in claim 16, wherein in step (c):
(i) At least a portion of the second intermediate stream is passed to a methane wash tower wherein said portion is contacted with methane to give an overheads stream comprising predominantly hydrogen and a bottoms stream comprising carbon monoxide and methane, and
(ii) the bottoms stream is then passed, with the third intermediate stream, to a carbon monoxide recovery process comprising a hydrogen stripper column and a carbon monoxide purification column.

26. A process as claimed in claim 25 wherein both the bottoms stream and the third intermediate stream are passed to the hydrogen stripper column, with the third intermediate stream being used as a reflux liquid stream and a bottoms stream from the hydrogen stripper column, rich in carbon monoxide and methane, is passed to the carbon monoxide purification column where the carbon monoxide and methane are separated as respective carbon monoxide-rich and methane-rich product streams.

27. A process as claimed in claim 16, wherein in step (c) the second and third intermediate streams are treated separately until they enter a common carbon monoxide purification column.

28. A process as claimed in claim 27, wherein:
(i) the second intermediate stream is passed to a rectification column where it is contacted with a reflux liquid stream comprising carbon monoxide which has itself been separated from the overhead stream from the rectification column to produce a first hydrogen-rich stream and a bottoms stream comprising predominantly methane and carbon monoxide, with some hydrogen, (ii) passing the bottoms stream from the rectification column to a hydrogen stripper column to recover a second hydrogen-rich stream and a first stream rich in carbon monoxide and methane, (iii) treating the third intermediate stream to remove hydrogen therein to produce a third hydrogen-rich stream and a second stream rich in carbon monoxide and methane, and (iv) passing the first and second streams rich in carbon monoxide and methane to a carbon monoxide purification column wherein the carbon monoxide and methane are separated as respective carbon monoxide-rich and methane-rich product streams.

29. A process as claimed in claim 16 wherein the process stream comprising carbon monoxide, hydrogen, methane and hydrocarbons with 2 or more carbons atoms is derived from the product stream of an autothermal cracking process for the production of olefins.

30. A process as claimed in claim 29 which process comprises:
(a) autothermally cracking a hydrocarbon-containing feedstock and an oxygen-containing gas in an autothermal cracking reactor over a supported catalyst capable of supporting combustion beyond the fuel rich limit of flammability to produce a product stream comprising olefins, unreacted hydrocarbons, hydrogen, carbon monoxide, methane, and small amounts of acetylenes, aromatics and carbon dioxide,
(b) quenching the reaction products in the product stream with water as they emerge from the autothermal cracking reactor,
(c) removing carbon dioxide from the product stream,
(d) passing all or a portion of this product stream to a first separation step to separate the product stream into a first intermediate stream comprising hydrocarbons with 2 or more carbons atoms and a reduced quantity of carbon monoxide, hydrogen and methane, and a second intermediate stream comprising carbon monoxide, hydrogen and methane and a reduced quantity of hydrocarbons with 2 or more carbons atoms,
(e) passing the first intermediate stream to a second separation step to separate the first intermediate stream into a product stream comprising hydrocarbons with 2 or more carbons atoms and a third intermediate stream comprising carbon monoxide, hydrogen and methane, and
(f) treating both the second and third intermediate streams in a cryogenic separation system to separate the carbon monoxide, and optionally the hydrogen, therein.
